(19) United States
(12) Patent Application Publication
(10) Pub. No.: US 2023/0248116 A1
(43) Pub. Date: Aug. 10, 2023

(54) REEL BASED CLOSURE SYSTEM

(71) Applicant: Boa Technology Inc., Denver, CO (US)

(72) Inventors: Josef Duller, Louisville, CO (US); Brendan Hoskens, Denver, CO (US); Blake Kittridge, Denver, CO (US); Mark Kerns, Golden, CO (US); Rebecca Linton, Denver, CO (US)

(73) Assignee: Boa Technology Inc., Denver, CO (US)

(21) Appl. No.: 18/091,935

(22) Filed: Dec. 30, 2022

Related U.S. Application Data
(60) Provisional application No. 63/295,395, filed on Dec. 30, 2021.

(51) Int. Cl. A43C 11/16

(52) U.S. Cl. CPC A43C 11/165

(57) ABSTRACT

A reel based closure device for tightening an article includes a housing and a spool that is positioned within the housing. The spool is rotatable within the housing to wind a tension member about a channel of the spool. A knob is supported by the housing and is operably coupled to the spool so that rotation of the knob causes the spool to rotate within the housing. A pawl component is operably couplable to the knob. The pawl component includes one or more pawls having a pawl arm fixedly connected at a proximal end to the pawl component. A distal end of the pawl arm includes one or more pawl teeth. The pawl component includes a lip and the pawl arm includes a tab that engages with the lip of the pawl component to control radial movement of the pawl arm relative to the pawl component.

### CROSS-REFERENCE TO RELATED APPLICATIONS

This application claims priority to Provisional U.S. Patent Application No. 63/295,395 filed Dec. 30, 2021, entitled “Reel Based Closure System,” the entire disclosure of which is hereby incorporated by reference, for all purposes, as if fully set forth herein.

### BACKGROUND OF THE INVENTION

The present disclosure is related to reel based closure devices for various articles, such as braces, medical devices, shoes, clothing, apparel, and the like. Such articles typically include some closure system, which allows the article to be placed about a body part and closed or tightened. The closure systems are typically used to maintain or secure the article about the body part. For example, shoes are typically placed over an individual's foot and the shoelace is tensioned and tied to close and secure the shoe about the foot. Similarly, braces are typically fit about the body and closed and tightened via hook and loop fasteners or similar mechanisms.

Conventional closure systems have been modified in an effort to increase the fit and/or comfort of the article about the body part. For example, shoe lacing configurations and/or patterns have been modified in an attempt to increase the fit and/or comfort of wearing shoes. Conventional closure systems have also been modified in an effort to decrease the time in which an article may be closed and secured about the body part. These modifications have resulted in the use of various pull cords, straps, and tensioning devices that enable the article to be quickly closed and secured about the wearer's body.

### BRIEF DESCRIPTION OF THE INVENTION

The embodiments described herein related generally to reel based closure devices and wearable braces. According to one aspect, a reel based closure device includes a housing, a spool rotatably positioned within the housing, and a knob that is operably coupled to the spool in a manner so that a rotation of the knob causes the spool to also rotate. The reel based closure device also includes a pawl component having one or more pawl arms that include a proximal end coupled to the pawl component and a distal end that includes one or more pawl teeth. The pawl component includes a lip or wall and the pawl arm includes a projection or tab that engages with the lip or wall of the pawl component to control radial movement of the pawl arm relative to the pawl component.

The pawl component is generally disc shaped, although other shapes may also be utilized. The lip or wall of the pawl component is generally formed or defined on outer peripheral edge of pawl component. The projection or tab of the pawl arm is typically formed on a radially interior edge of pawl arm. In a specific instance, the projection or tab is positioned on the distal end of the pawl arm. The one or more pawl teeth of the pawl arms are configured to engage with teeth of the reel based closure device when the knob is in an engaged position. When the knob is in a disengaged position, the one or more pawl teeth disengage from the teeth of the reel based closure device. The knob is commonly moveable axially with respect to the housing between the engaged position and the disengaged position. The proximal end of the pawl arm is typically fixedly coupled or attached to the pawl component. The pawl component is typically a separate component from the knob.

A method of assembling a reel based closure device may include obtaining a housing and positioning a spool within the housing so that the spool is rotatable within the housing. The method may also include coupling a pawl component with the housing and coupling a knob with the housing so that the knob is operably coupled to the spool. The pawl component may include one or more pawl arms having a proximal end that is coupled to the pawl component and a distal end that includes one or more pawl teeth. The pawl component may include a lip or wall and the pawl arm may include a projection or tab that engages with the lip or wall of the pawl component to control radial movement of the pawl arm relative to the pawl component.

In some instances, the method may further include positioning the projection or tab of the pawl arm radially inward of the lip or wall of the pawl component so that a radially outward movement of the pawl arm engages the projection or tab of the pawl arm with the lip or wall of the pawl component. The method may additionally include axially inserting the pawl component into a bottom end of the knob and/or attaching the reel based closure device to an article of footwear.

According to another aspect, a spool for a reel based closure device includes an upper flange, a lower flange, and a central body that is formed, positioned, or defined between the upper flange and the lower flange. The central body may be configured so that a tension member is windable about the central body when the spool is rotated in a first direction and is unwindable from the central body when the spool is rotated in a second direction. A channel or recess extends into the central body. The channel or recess may extend through the central body from one side of the central body to an opposite side of the central body. A cross-sectional profile of the central body is non-circular or asymmetrical, in which the cross-sectional profile is taken or viewed along a plane that is orthogonal to a central axis of the spool. The plane may intersect the central body roughly centrally along the central axis of the spool.

In some instances, the includes a single channel or recess. In other instances, the channel or recess may be a first channel or recess and the spool may include a second channel or recess. The first channel or recess and the second channel or recess may be positioned on opposite sides of a central aperture of the spool. The central body of the spool may have an oval shaped profile. The cross-sectional profile of the central body may have a linear portion or segment. In some instances, the linear portion or segment may be a first linear portion or segment and the cross-sectional profile may have a second linear portion or segment that is separate from the first linear portion or segment. The second linear portion or segment may have a shorter length than the first linear portion or segment. A majority of the cross-sectional profile of the central body may be circular. A portion of the cross-sectional profile of the central body that is adjacent to the channel may have a triangular shape.

A method of assembling a reel based closure device may include obtaining a spool having a non-circular or asymmetrical shape as described above and inserting the spool into a housing of the reel based closure device so that the spool is rotatable within the housing. The method may also include coupling a tension member to the channel or recess to attach the tension member to the spool. Coupling the tension member to the channel or recess may include inserting the tension member through the channel or recess, tying a knot in the tension member or attaching a stop component to the tension member, and retracting the stop component into the channel or recess. The method may additionally include attaching the reel based closure device to an article of footwear.

According to another aspect, a reel based closure device includes a housing, a spool, a knob that is rotatably coupled with the housing, and a drive component that is operably coupled with the knob and spool so that a rotation of the knob causes the spool to rotate in a tightening direction. The spool includes a channel about which a tension member is wound as the spool is rotated in the tightening direction. The spool also includes a plurality of teeth that are configured to engage a plurality of teeth of the drive component. The teeth of the spool and drive component typically extend axially in opposing directions. An angle of a mating surface or face of each tooth of the spool or drive component is varied along a length of the respective tooth.

The mating surface or face of each tooth of the spool or drive component may include a first portion or area and a second portion or area. The first portion or area may be angled relative to the second portion or area. For example, the second portion or area may be more vertically oriented than the first portion or area with respect to a central axis of the reel based closure device. In some instances, an angle of a mating surface or face of each tooth of the spool and an angle of a mating surface or face of each tooth of the drive component is varied along the length of the respective tooth so that the teeth of both the spool and drive component have varying angles along the length of the respective teeth.

The plurality of teeth of the spool may be positioned on an upper surface of the spool and the plurality of teeth of the drive component may be positioned on a lower surface of the drive component. The drive component may be axially moveable with respect to the spool between an engaged position, in which the teeth of the drive component are engaged with the teeth of the spool, and a disengaged position, in which the teeth of the drive component are disengaged from the teeth of the spool. The reel based closure device may also include a plurality of pawls that are configured to engage with teeth of the reel based closure device to prevent rotation of the spool in the loosening direction. Each pawl of the plurality of pawls may include a pawl arm having a proximal end that is coupled to the drive component and a distal end that includes one or more pawl teeth.

A method of assembling a reel based closure device includes providing a housing, positioning a spool within the housing, operably coupling a drive component with the spool, and coupling a knob with the housing so that a rotation of the knob causes the spool to rotate in a tightening direction. As described above, an angle of a mating surface or face of each tooth of the spool or drive component may be varied along a length of the respective tooth. The method may also include attaching the reel based closure device to an article of footwear.

According to another aspect, a reel based closure device includes a housing having an interior wall and a plurality of channels or recesses that are formed or defined in the interior wall, a spool that is positioned within the housing, and a knob that is operably coupled to the spool to effect rotation of the spool in a tightening direction. The spool includes a channel about which a tension member is wound as the spool is rotated in the tightening direction within the housing. The plurality of channels or recesses that are formed or defined in the interior wall of the housing extend axially along the interior wall and define surfaces that are positioned between adjacent channels. The spool contacts one or more of the defined surfaces, at least temporarily, as the spool is rotated in the tightening direction within the housing.

The housing may have an open bottom end. In such instances, each channel or recess may extend to the open bottom end of the housing. The housing may include an annular rib that is formed or defined on the interior wall between an upper portion and a lower portion of the housing. In such instances, each channel or recess may extend to the annular rib. The reel based closure device may include between 4 and 20 channels or recesses. The reel based closure device may also include a pawl component that includes one or more pawls. The pawl component may be configured to prevent rotation of the spool in a loosening direction. The reel based closure device may additionally include a base that is positioned axially below the spool and that is releasably attachable to a bottom end of the housing.

A method of assembling a reel based closure device includes providing a housing having an interior wall and a plurality of channels or recesses that are formed in the interior wall. A spool is positioned within the housing and a knob is operably coupled with the housing and spool so that a rotation of the knob causes the spool to rotate in a tightening direction. As described above, the plurality of channels or recesses extend axially along the interior wall of the housing and define surfaces that are positioned between adjacent channels. The spool contacts one or more of the defined surfaces, at least temporarily, as the spool is rotated in the tightening direction within the housing. The method may also include attaching the reel based closure device to an article of footwear.

According to another aspect, a strap for use with a reel based closure device includes a proximal end, a distal end, a mid-section that is positioned between the proximal and distal ends, and a window or elongate channel that is formed or defined in the mid-section of the strap. The window or elongate channel is positionable over the reel based closure device in a manner so that the strap is moveable with respect to the reel based closure device while the reel based closure device is fully surrounded by the mid-section of the strap.

The window or elongate channel typically has a longitudinal length that is greater than a length or diameter of the reel based closure device. The window or elongate channel typically has a lateral width that is greater than a width or diameter of the reel based closure device. The strap may be moveable longitudinally relative to the reel based closure device while the reel based closure device is fully surrounded by the mid-section of the strap. The mid-section of the strap may be reinforced with respect to the proximal and distal ends of the strap. In a specific instance, a reinforcing material or layer is coupled with the mid-section of the strap at or near the window or elongate channel. The proximal end and/or the distal end of the strap may be formed of an elastic or semi-elastic material. The strap may also include a fastening mechanism that enables fastening of the strap to another strap or other material. Specific examples of fastening mechanisms include a hook and loop fastener, a clamp, a buckle, a snap, a magnetic fastener, and the like, or a combination thereof. The strap may be used to close and/or tighten an article. A specific example of an article that may particularly be suited for the strap is a brace, such as an ankle brace. The strap may be attached to a bottom end of an ankle brace.

A method of manufacturing a brace includes providing a brace, attaching a reel based closure device to the brace, and coupling a strap to the brace. As described above, the strap includes a proximal end, a distal end, a mid-section that is positioned between the proximal and distal ends, and a window or elongate channel formed or defined in the mid-section of the strap. The window or elongate channel is positionable over the reel based closure device so that the strap is moveable about the brace and with respect to the reel based closure device while the reel based closure device is fully surrounded by the mid-section of the strap.

In a specific instance, the brace is an ankle brace. In such instances, coupling the strap with the brace may include attaching the strap to a bottom end of the ankle brace so that a portion of the strap is positioned below a wearer's foot when the ankle brace is positioned about the wearer's foot.

### BRIEF DESCRIPTION OF THE DRAWINGS

The present invention is described in conjunction with the appended figures:

FIGS. 1A-5 illustrate a reel based closure device that may be used to tension a lace or tension member and thereby close and/or tighten an article or other item.

FIGS. 6A-C illustrate a first embodiment of a knee brace that includes a petal configuration that is positionable around the patella.

FIGS. 7A-C illustrate a second embodiment of a knee brace that includes a petal configuration that is positionable around the patella.

FIGS. 8A-D illustrate a third embodiment of a knee brace that includes a petal configuration that is positionable around the patella.

FIG. 9 illustrates a fourth embodiment of a knee brace that is configured to surround and support the patella.

FIGS. 10A-C illustrate a fifth embodiment of a knee brace that includes a support member for supporting and/or cushioning the patella.

FIGS. 11A-D illustrate an embodiment of an ankle brace that may be fit about the ankle to support and strengthen the ankle.

FIGS. 12A-G illustrate another embodiment of an ankle brace that may be fit about the ankle to support and strengthen the ankle.

In the appended figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DETAILED DESCRIPTION

The ensuing description provides exemplary embodiments only, and is not intended to limit the scope, applicability or configuration of the disclosure. Rather, the ensuing description of the exemplary embodiments will provide those skilled in the art with an enabling description for implementing one or more exemplary embodiments. It being understood that various changes may be made in the function and arrangement of elements without departing from the spirit and scope of the invention as set forth in the appended claims.

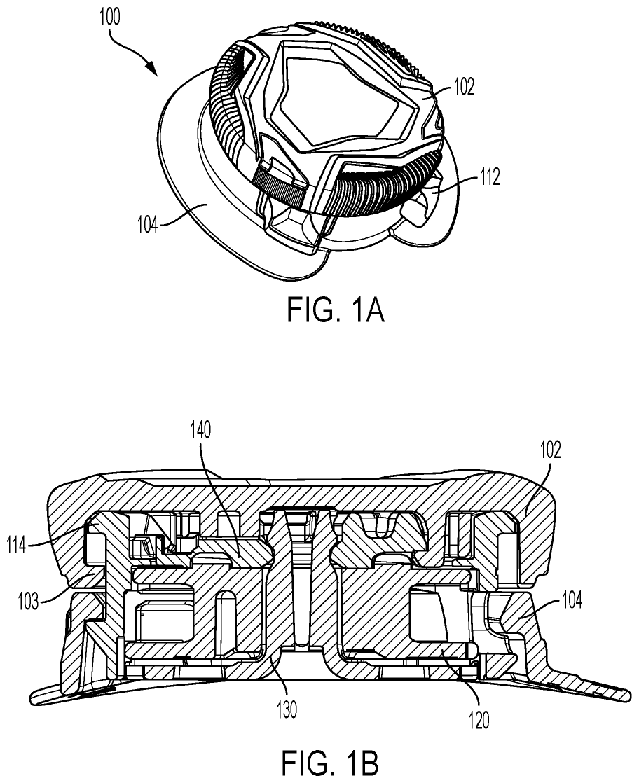

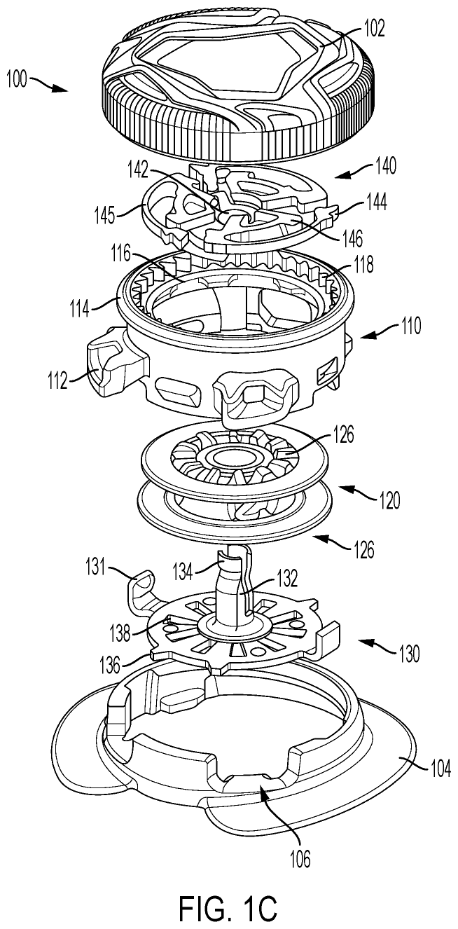

The embodiments of FIGS. 1A-5 provide reel based closure devices (hereinafter closure system or reel based device/system) that may be used to tension a lace or tension member and thereby tighten an article or other item. The article may be a variety of items including a pack (i.e., back pack, book bag, etc.), an article of clothing (i.e., hats, gloves, belt, etc.), sports apparel (boots, snowboard boots, ski boots, etc.), medical braces (i.e., back braces, knee braces, wrist brace, ankle brace, etc.), and various other items or apparel. A specific embodiment in which the closure system may be employed involves footwear, such as shoes, boots, sandals, etc.

Referring to FIGS. 1A-5, illustrated is an embodiment of a reel based closure device 100 that may be used to tighten an article, such as a shoe, boot, or other article of footwear. The reel based closure device 100 may also be used to tighten other non-footwear articles, such as backpacks, helmets, jackets, etc. The reel based closure device 100 may be used to tighten essentially anything that is closed and/or tightened via a tension member, cord, lace, rope, and the like. FIG. 1A illustrates an assembled view of the reel based closure device 100 while FIGS. 1B illustrates a cross section view of the reel based closure device and FIG. 1C illustrates an exploded perspective view of the reel based closure device 100. Various internal components are illustrated in the views of FIGS. 1B and 1C.

The reel based closure device 100 includes a housing 110 that has an interior region within which various components of the reel based closure device 100 are positioned. For example, a spool 120 is positioned within the interior region of the housing 110. The spool 120 is rotatable within the interior region of the housing in a first direction to wind a tension member (not shown) about the spool and is also rotatable in a second direction within the interior region of the housing 110 to unwind the tension member from about the spool 120. The spool 120 typically includes an annular channel, or central cylindrical body, about with the tension member is wound and unwound. A knob 102 is operably coupled with the spool 120 and with the housing 110. The knob 102 is user rotatable in a tightening direction to effect or cause the spool 120 to rotate in the first direction and thereby wind the tension member about the spool 120. In some embodiments, the knob is also rotatable in a loosening direction to effect or cause the spool 120 to rotate in the second direction and thereby unwind the tension member from about the spool 120. The tightening direction typically is the same rotational direction as the first direction (e.g., clockwise), but in some instances, the tightening direction may be an opposite direction of the first direction. Likewise, the loosening direction is typically the same rotational direction as the second direction (e.g., counterclockwise), but in some instances, the loosening direction may be opposite of the second direction. For convenience in describing the reel based closure device 100, the spool 120 will hereinafter be described as being rotatable in the tightening direction to wind the tension member and as being rotatable in the loosening direction to unwind the tension member. Other components of the reel based closure device 100 include the pawl disc 140, the seat component 130 (also referred to herein as a base component), and the base member 104.

The housing 110 is positionable within the base member 104 or bayonet (hereinafter base member 104) that is attachable to the article. When the housing 110 is coupled with the base member 104, a pair of lace ports 112 of the housing 110 are positioned within corresponding openings or windows 106 of the base member 104. Positioning of the lace ports 112 within the openings 106 of the base member 104 allows the reel based closure device 100 to have a more seamless appearance. The lace ports 112 provide interior access to the tension member, which allows the tension member to access the spool so that the tension member is windable and unwindable about the spool. The housing 110 also includes an inner annular ridge 116 that encircles the interior region of the housing 110 and partitions the interior region into an upper portion and a lower portion. In some embodiments, the inner annular ridge 116 functions to prevent the components that are positioned in the lower portion from moving into the upper portion of the housing and to prevent the components that are position in the upper portion from moving into the lower portion of the housing.

The housing 110 further includes depressions or teeth 118 that are configured to engage with one or more teeth of the pawl disc 140. The teeth 118 may be formed on the inner surface of the housing 110 so that the teeth 118 are radially inward facing as illustrated in FIG. 1C. In other instances, the teeth 118 may be formed on an upper surface of the housing 110 so that the teeth 118 are axially upward facing (not shown). In yet other instances, the teeth 118 may be formed on a separate component (not shown) that is couplable with the housing 110. The teeth 118 may face axially or radially on the separate component as desired. The teeth 118 may have a triangular shaped configuration or may be formed of voids or apertures similar to the teeth 138 of the bottom component or seat component 130 (hereinafter seat component 130).

The housing 110 includes a coupling feature that enables the housing 110 to snap fit couple with the knob 102. In one instance, the housing's coupling feature is an annular ridge or ring 114 that extends partially or fully around the upper surface of the housing 110. The knob 102 includes one or more radially inward protruding tabs 103 that are configured to snap over the housing's annular ridge 114 in order to attach the knob 102 to the housing 110. The annular ridge 114 has a diameter that is larger than an inner diameter that is defined by the one or more radially inward protruding tabs 103. As such, when the knob 102 is axially aligned with the housing 110 and the knob 102 is pressed axially downward about the housing 110, the knob 102 is forced to flex or slightly bend radially outward, which enables the knob 102 to be moved axially downward relative to the housing 110 and allows the radially inward protruding tabs 103 to be positioned under the annular ridge 114 as illustrated in FIG. 1B. A top end of the annular ridge 114 and a bottom end of the one or more radially inward protruding tabs 103 is chamfered or angled, which aids in snap fit coupling the knob 102 and housing 110 as described herein. The larger diameter annular ridge 114 prevents the knob 102 from uncoupling or detaching from the housing 110. The snap fit coupling of the knob 102 and housing 110 allows the reel based closure device 100 to be coupled without requiring the use of a bolt, screw, rivet, or other similar mechanical fastener. Additional details of the coupling of the knob 102 and housing 110 are provided in U.S. patent application Ser. No. 16/864,849, filed May 1, 2020, entitled “Reel Based Closure System,” the entire disclosure of which is incorporated by reference herein.

The seat component 130 that is positioned axially below the spool 120 and is releasably coupled with a bottom end of the housing 110. The seat component 130 typically includes a circular base and a central boss or protrusion 132 that protrudes axially upward from the circular base into the interior region of the housing 110 when the seat component 130 is coupled with the bottom end of the housing 110. The circular base functions as the bottom surface of the housing 110 when the seat component 130 is attached to the housing 110. The circular base matches an opening in the bottom end of the housing 110 so that when the seat component 130 is coupled with the housing 110, the bottom end of the housing 110 is fully covered and enclosed by the circular base. The central boss 132 is configured to be inserted through an aperture of the spool 120. The central boss 132 is also axially insertable through a central aperture 142 of the pawl disc 140. The central boss 132 and the pawl disc 140 operably engage to allow the reel based closure device 100 to be moved between an engaged state and a disengaged state.

The spool 120 is rotationally positioned about the seat component 130 by inserting the central boss 132 through the spool's central aperture. The spool 120 is able to spin or rotation about the central boss 132 with negligible friction. The seat component 130 includes a plurality of radially extending fingers 136 that are configured for positioning in corresponding recesses (not shown) on the bottom end of the housing 110. Insertion of the radially extending fingers 136 within the recesses rotationally locks the seat component 130 relative to the housing 110 by preventing relative rotation of the two components. The seat component 130 also includes a pair of coupling arms 131 that are positioned on opposing sides of the seat component 130 and that couple the seat component 130 with the housing 110. The coupling arms 131 extend radially from the seat component 130 and releasably engage the bottom end of the housing 110 in order to releasably couple the seat component 130 with the bottom end of the housing 110. The coupling arms 131 include an upward turned lip that is configured to flex, bend, or curve around or over a bottom edge of the housing 110 to secure the seat component 130 to the bottom end of the housing 110. The bottom end of the housing 110 includes a pair of small channels within which the coupling arms 131 are positioned so that when the seat component 130 is coupled with the housing 110, a bottom surface of the seat component 130 is aligned with a bottom surface of the housing 110.

Each upward turned lip includes a radially inward facing protrusion that is designed for positioning within a small recess on the exterior of the housing 110. Positioning of each protrusion within the corresponding recess secures the seat component 130 to the housing 110. The seat component 130 may be detached from the housing 110 by flexing or bending the upward turned lip radially outward so that the protrusion is removed from the housing's recess. An axial downward force may simultaneously be applied to the seat component 130 to cause the seat component 130 to move axially downward relative to the housing 110.

The pawl disc 140 typically includes a plurality of pawl arms 145 that are attached to a central body or base 146 of the pawl disc 140. In the illustrated embodiment, the pawl disc 140 includes three pawl arms 145, although in other embodiments the pawl disc 140 may include more or fewer pawl arms 145 as desired (e.g., a single pawl arm, two pawl arms, four pawl arms, and the like). Each pawl arm 145 is fixedly attached to the central body 146 at a proximal end of the respective pawl arm 145. Each pawl arm 145 also includes or more teeth 144 positioned at a distal end of the respective pawl arm 145. The one or more pawl teeth 144 are configured to engage the teeth 118 of the housing 110 in a ratchet like manner in order to allow a one way rotational movement of the pawl disc 140 and spool 120 relative to the housing 110. Engagement of the one or more pawl teeth 144 with the housing's teeth 118 locks the pawl disc 140 and spool 120 in rotational position relative to the knob 102 and housing 110. The spool 120 is locked rotational position relative to the housing 110 due to engagement of the spool's teeth 126 and the pawl disc's teeth 147. Each pawl arm 145 functions as a spring member that biases the one or more pawl teeth 144 radially outward and into engagement with the teeth 118 of the housing 110. The pawl arm 145 may also flex radially outward into contact with the teeth 118 of the housing 110, which may reinforce the locked engagement of the pawl disc 140 and housing 110.

The pawl disc 140 may be snap together coupled with the knob 102. To couple the pawl disc 140 with the knob 120, the knob may include one or more axially extending tabs (not shown) that snap into engagement with the central body 146 of the pawl disc 140. For example, a distal end of the knob's axially extending tab may include a lip that is shaped and sized to be positioned within a recess or pocket of the pawl disc's central body 146. To couple the knob 102 and the pawl disc 140, the pawl disc 140 is axially aligned with the knob 102 and the pawl disc 140 is then be pressed axially upward into the interior of the knob 102, which causes the knob's axially extending tabs to flex radially outward and around the main body of the pawl disc 140.

The pawl disc 140 is operationally engaged with the spool 120, which prevents the spool 120 from rotating in the loosening direction. The force or load that is exerted on the pawl arms 145 from tension in the tension member is transferred to the one or more pawl teeth 144, which engage with the teeth 118 of the housing 110 and thereby prevent rotation of the spool 120. The pawl disc 140 is also operationally engaged with the knob 102 such that when the knob 102 is rotated in the tightening direction, the pawl disc 140 is also rotated in the tightening direction. Rotation of the pawl disc 140 in the tightening direction causes the one or more pawl teeth 144 to deflect radially inward and incrementally out of engagement with the teeth 118. The pawl arms 145 bias the one or more pawl teeth 144 back into engagement with the teeth 118 as rotation of the pawl disc 140 is ceased, or as the pawl disc 140 is rotated in the loosening direction via tension in the tension member. The operational engagement of the pawl disc 140 and spool 120 results in rotational forces imparted on the knob 102 being transferred to the spool 120, which causes the spool 120 to rotate within the housing 110 in the tightening direction. Rotation of the spool 120 in the tightening direction causes the tension member to be wound about the spool 120.

In some instances, the one or more pawl teeth 144 may be oriented to engage with axially oriented teeth 118. In such embodiments, the one or more pawl teeth 144 may displace or move axially in response to rotation of the pawl disc 140 in the tightening direction.

The pawl disc 140 is operationally engaged with the central boss 132 in a manner that enables the pawl disc 140 and/or knob 102 to be supported in one of two positions: an engaged position and a disengaged position. In the engaged position, the pawl disc 140 and/or knob 102 are positioned axially downward with respect to the housing 110 and spool 120, which enables the knob 102 to be operated to tighten the tension member as described herein. FIG. 1B illustrates the pawl disc 140 and knob 102 in the engaged position. In the disengaged position, the pawl disc 140 and/or knob 102 are positioned axially upward with respect to the housing 110 and spool 120, which enables the spool 120 to freely spin in the loosening direction to freely loosen the tension member's tension.

In the disengaged position, the axially oriented teeth 126 of the spool 120 are disengaged from, and do not contact, the axially oriented teeth 147 of the pawl disc 140. Since the axially oriented teeth, 126 and 147, of the spool 120 and pawl disc 140 are disengaged, the spool 120 is able to spin or rotate freely within the housing 110 in the loosening direction. In the disengaged position, the one or more pawl teeth 144 of the pawl disc 140 may disengage from the housing's teeth 118, which may allow the knob 102 and/or pawl disc 140 to be rotated in the loosening direction. In other embodiments, the one or more pawl teeth 144 may remain engaged with the housing's teeth 118 in the disengaged position, which may prevent rotation of the knob 102 and/or pawl disc 140 in the loosening direction.

In some embodiments, axially upward movement of the pawl disc 140 into the disengaged position may be achieved by pulling axially upward on the knob 102. When the knob 102 is moved axially upward relative to the housing 110, the radially inward protruding tabs 103 of the knob 102 may contact the housing's annular ridge 114. In other embodiments, the knob 102 may remain axially stationary with respect to the housing 110 and spool 120 while the pawl disc 140 is moved axially upward into the disengaged position. In such embodiments, axial upward movement of the pawl disc 140 may be achieved by rotating the knob 102 in the loosening direction and/or by employing a separate release mechanism or button, such as a lever, button, clamp, and the like. For example, to move the pawl disc 140 axially upward, the knob 102 and/or pawl disc 140 may include cammed, ramped, or sloped surfaces, or another mechanism, which moves the pawl disc 140 axially upward as the knob 102 is rotated in the loosening direction.

FIG. 1B illustrates the engaged position of the reel based closure device 100 in which the pawl disc 140 is positioned axially downward relative to the housing 110 and spool 120. In the engaged position, the spool's axially oriented teeth 126 are engaged with, and directly contact, the pawl disc's axially oriented teeth 147. Engagement of the axially oriented teeth, 126 and 147, of the spool 120 and pawl disc 140 operationally locks the pawl disc 140 and spool 120 together so that a rotation of the pawl disc 140 in the tightening direction causes the spool 120 to rotate in the tightening direction. The spool 120 and pawl disc 140 are also operationally locked so that a rotation of the spool 120 in the loosening direction causes the pawl disc 140 to rotate in the loosening direction. In the engaged position, the pawls 145 of the pawl disc 140 are engaged with the housing's teeth 118, which locks the pawl disc 140 and spool 120 in rotational position relative to the housing 110 as described herein. The knob 102 is likewise positioned axially downward with respect to the housing 110 and spool 120 in the engaged position.

The central boss 132 supports and maintains the pawl disc 140 and/or knob 102 in the engaged and disengaged positions via an annular projection or member 134. The annular member 134 has a diameter that is greater than the diameter of the pawl disc's central aperture 142, which causes the annular member 134 to interfere with and impede axially upward and downward movement of the pawl disc 140 about the top end of the central boss 132. While the annular member 134 impedes axial movement of the pawl disc 140, the annular member 134 does not prevent axial movement of the pawl disc 140 due to the ability of the central boss 132 to displace or flex radially inward. Specifically, the central boss 132 is formed of a pair of fingers or protrusions that extend axially upward from the circular base of the seat component 130. The pair of protrusions flex radially inward toward one another as the pawl disc 140 is moved axially upward and downward in relation to the annular member 134. Specifically, the engagement of the annular member 134 and the pawl disc's central aperture 142 forces the pair of protrusions to flex inward as the pawl disc 140 is moved axially upward and downward about the annular member 134. After the pawl disc 140 is moved axially upward or downward about the annular member 134, the pair of protrusions resiliently flex outward to resume an un-deflected configuration. In operation, the pawl disc's central aperture 142 is positioned above or below the annular member 134, which supports and maintains the pawl disc 140 and/or knob 102 in either the engaged or disengaged position.

The reel based closure device 100 may include a reinforcement spring (not shown) to strengthen and reinforce the pair of protrusions of the seat component 130. To reinforce the pair of protrusions, the reinforcement spring is inserted axially within an axially extending gap between the pair of protrusions of the seat component 130. The reinforcement spring is made of a flexible and resilient material, such as spring steel or a metal free (e.g., PEAK) material. The reinforcement spring aids in resiliently deflecting the pair of protrusions as the pawl disc 140 is moved axially upward and downward about the annular member 134. The reinforcement spring may also stiffen the pair of protrusions and prevent the pair of protrusions from plastically deforming due to extended use of the reel based closure device 100.

As illustrated in FIG. 1C, the seat component 130 includes a spool engagement feature 138 that is configured to engage a bottom end of the spool 120 as a tension in the tension member decreases. Typically the spool engagement feature 138 and the spool 120 do not engage until a tension in the tension member is at or near a tension threshold, such as a nominal or zero amount of tension. Engagement of the bottom end of the spool 120 and the spool engagement feature 138 prevents rotation of the spool 120 in the loosening direction. Stated differently, the spool 120 is rotatable in the loosening direction until the bottom end of the spool 120 contacts and engages with the spool engagement feature 138. After engagement of the spool 120 and the spool engagement feature 138, rotation of the spool 120 in the loosening direction is prevented.

In one embodiment, the spool 120 includes teeth (not shown) that are positioned on and axially extend from a bottom surface of the spool 120. The spool's teeth are configured to engage with circumferentially spaced apertures that are formed on the seat component's circular base. The apertures function as the spool engagement feature 138 (hereinafter apertures 138) such that engagement of the spool's teeth with the circumferentially spaced apertures prevents rotation of the spool 120 in the loosening direction. In other embodiments, the spool engagement feature may be teeth that are formed on the circular base, or may be other frictional components, such as a rubber type gasket or material, abrasive materials, tacky materials, and the like.

The spool's teeth and the apertures 138 are disengaged until the tension member's tension is at or near the tension threshold. Disengagement of the spool's teeth and apertures 138 allows the spool 120 to rotate in the tightening and loosening direction as described herein to tension and loosen the tension member. After the spool's teeth and apertures 138 engage, further rotation of the spool 120 in the loosening direction is prevented or limited. To allow engagement and disengagement of the spool's teeth and the apertures 138, the spool 120 is configured to move axially upward and downward relative to housing 110.

The engagement of the spool's teeth 126 and the pawl disc's teeth 147 causes both the spool 120 to move axially upward when the pawl disc 140 is rotated in the tightening direction. The spool's teeth 126 and the pawl disc's teeth 147 engage when some amount of tension exists in the tension member since the tension will urge the spool 120 to rotate in the loosening direction relative to the pawl disc 140, which will cause the spool's teeth 126 to slide axially upward relative to and into further engagement with the pawl disc's teeth 147 due to the shape of the taper/slope of the teeth, 126 and 147.

As the tension in the tension member is decreased, at some point the tension will achieve or exceed the tension threshold, which will cause the spool 120 to move axially downward relative to housing 110 and will cause the spool 120 to contact and engage with the seat component's teeth 138. Frictional engagement of the spool 120 and seat component's teeth 138 prevents or limits further rotational movement of spool 120 in the loosening direction. Rotation of the spool 120 in the loosening direction is prevented even if and when the knob 102 is rotated in the loosening direction. The spool 120 will remain in the axially downward position until the pawl disc 140 is rotated in the tightening direction, which will cause the spool 120 to move axially upward as previously described.

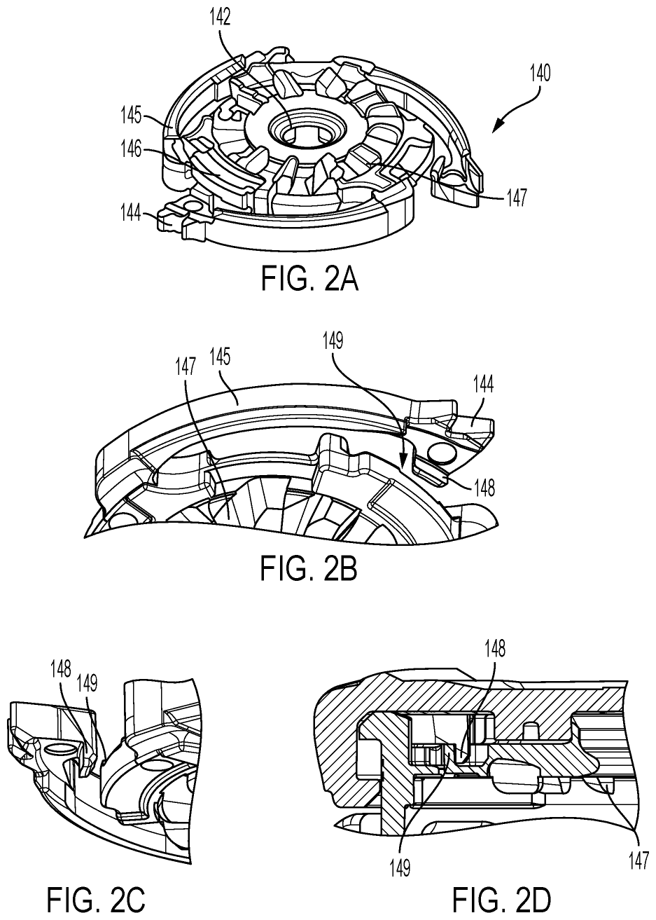

FIGS. 2A-D illustrate the pawl disc 140 in greater detail. FIG. 2A illustrates the bottom end of pawl disc 140 so that the axially oriented teeth 147 are visible. The pawl arms 145, one or more pawl teeth 144, central body 146, and central aperture 142 are also clearly visible. As further illustrated in FIGS. 2A-B, the distal end of the pawl arms 145 includes a clip or tab 148 (hereinafter clip 148) that extends axially downward from a bottom surface of the pawl arms 145, roughly in the same direction as the teeth 147. The clip 148 is positioned on a side of the distal end of the pawl arms 145 opposite the one or more teeth 144. The clip 148 is configured to engage a small lip or wall 149 (hereinafter wall 149) that is formed on an outer edge of the central body 146 of the pawl disc 140. The wall 149 is visible in FIG. 2D.

As illustrated in FIG. 2D, the clip 148 is positionable on an inner surface of the wall 149 within a recess or channel that is formed on the outer edge of the central body 146. The clip 148 is roughly the same height as the wall 149 and engages with the inner surface of the wall 149. Engagement of the clip 148 and wall 149 prevents the clip 148 from moving radially outward of the wall 149, which restrains the outward radial movement of the respective pawl arm 145. More specifically, each pawl arm 145 is able to move radially outward until its respective clip 148 contacts and engages the respective wall 149 of the central body 146. Once the clip 148 and wall 149 engage, further outward radial movement of the respective pawl arm 145 is prevented. One function of the clips 148 and walls 149 may be to maintain the pawl arms 145 in an ideal orientation for assembly of the pawl disc 148 with the knob 102 and housing 110. Another function of the clips 148 and walls 149 may be to facilitate in reengagement of the pawl disc's one or more teeth 144 and the housing's teeth 118 as the pawl disc 140 is moved or transitioned between the disengaged position and the engaged position.

The clips 148 and walls 149 are arranged so that when the pawl disc 140 is inserted within the housing 110 and is in the engaged position, the pawl disc's one or more teeth 144 engage with the housing's teeth 118. Stated differently, the clips 148 and walls 149 to not interfere with, hamper, or impede engagement of the pawl disc's one or more teeth 144 engage and the housing's teeth 118 during use of the reel based closure device 100. In some embodiments, the clips 148 and walls 149 may not engage after the pawl disc 140 is assembled with the housing 110 and knob 102. In other embodiments, the clips 148 and walls 149 may temporarily engage when the pawl disc 140 is moved axially upward into the disengaged position previously described. In such embodiments, the clips 148 and walls 149 may facilitate in reengagement of the pawl disc's one or more teeth 144 and the housing's teeth 118 by restraining the radial outward movement of the pawl arms 145 during disengagement of the pawl disc 140 and spool 120. The clips 148 and walls 149 may facilitate in reengagement of the pawl disc's one or more teeth 144 and the housing's teeth 118 by maintaining the pawl arms 145 in a radial orientation relative to the central body 146 and housing 110 that enables the pawl disc's one or more teeth 144 to easily slide or move axially into the housing's teeth 118.

In some embodiments, an outer surface or edge of the clip 148 may have a chamfered or angled orientation (see FIG. 2D) that aids in moving the clip 148 radially inward and over the wall 149. In such embodiments, the outer surface of the wall 149 may have a corresponding chamfered or angled orientation. The clip 148 is typically formed on a tab or nub that extends radially inward from a main body of the distal end of the respective pawl arm 145. Positioning of the clip 148 on the radially extending tab or nub enables a desired degree of radially outward movement of the distal end of the pawl arm 145 to be achieved.

The clips 148 and walls 149 may enable stronger pawl arms 145 to be employed in the reel based closure device 100. The stronger pawl arms 145 may be formed by thickening a wall of the pawl arm 145, by using more resilient materials, and/or by coupling a reinforcement spring or material with the pawl arm 145. The strengthened pawl arms 145 may enable the reel based closure device 100 to handle or accommodate greater tensile loads in the tension member. The clips 148 and walls 149 may prevent the strengthened pawl arm 145 from moving or springing outward in a manner that would damage the reel based closure device 100, such as the housing's teeth 118, and/or that would impede reengagement of the pawl disc's one or more teeth 144 and the housing's teeth 118 as the pawl disc 140 is moved or transitioned into the engaged position. The clips 148 and walls 149 may also reduce an overall height of the reel based closure device 100 by allowing one or more internal components or features to be omitted. The reduced height of the reel based closure device 100 may allow the reel based closure device to be used in a wider variety of applications and/or reduce unwanted contact of the reel based closure device 100 with external objects.

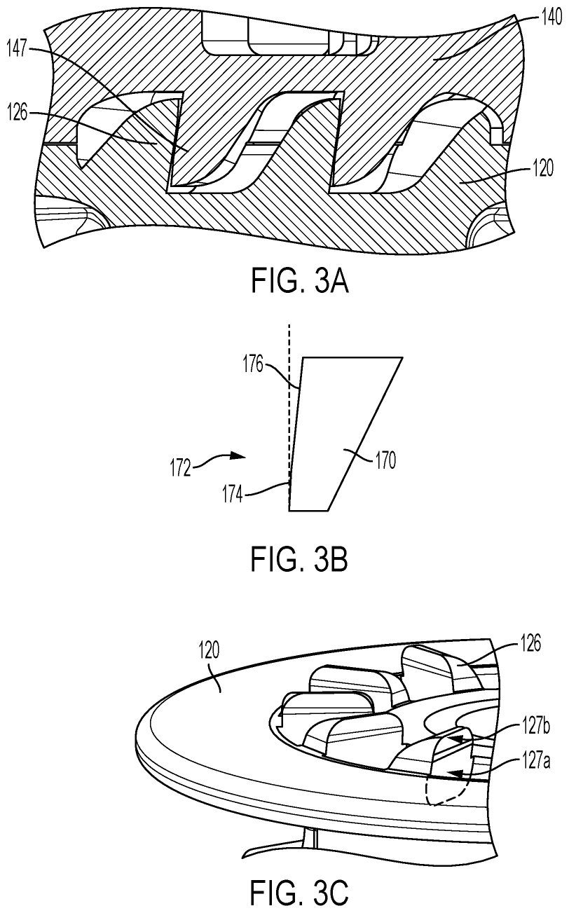

FIGS. 3A-C illustrate the pawl disc teeth 147 and the spool teeth 126 in greater detail. FIG. 3A illustrates engagement of the pawl disc teeth 147 and spool teeth 126. The pawl disc teeth 147 and/or spool teeth 126 extend axially in opposing directions and are designed to facilitate engagement of the teeth as the pawl disc 140 is moved or transitioned between the disengaged and engaged positions described herein. Easy engagement of the pawl disc teeth 147 and spool teeth 126 may be important when the knob 102 is contacted by external object in a manner that causes the knob 102 to pivot or tilt to some degree. Without easy engagement of the pawl disc teeth 147 and spool teeth 126, such contact may cause the knob 102 to unintentionally move upward, thereby releasing tension on the tension member. This unwanted action (i.e., unintentional loosening of the tension member) may be greatly reduced when the pawl disc teeth 147 and spool teeth 126 are able to easily reengage.

To facilitate engagement of the pawl disc teeth 147 and spool teeth 126, a profile or shape of an engaging or mating surface, or face, of each pawl disc tooth 147 and/or spool tooth 126 may not be linear or straight. Rather, the engaging surface, or face, of each pawl disc tooth 147 and/or spool tooth 126 may be configured so that a taper/slope, or angle, of the engaging surface varies in comparison with a vertical reference line. Stated differently, an angle of the mating surface or face of each pawl disc tooth 147 and/or spool tooth 126 is varied along an axial length of the respective tooth. More specifically, the distal portion or tip of the engaging surface/face of each pawl disc tooth 147 and/or spool tooth 126 may be more vertically oriented in comparison with a proximal portion of the engaging surface/face of each pawl disc tooth 147 and/or spool tooth 126.

FIG. 3B illustrates an exemplary tooth 170 having an engaging surface or face 172, in which an angle of the engaging surface or face 172 is varied along a length of the tooth 170. The tooth 170 may be representative of each pawl disc tooth 147 and/or each spool tooth 126. As illustrated, the tooth 170 includes a distal portion or section (also referred to herein as a second angled portion) 174 and a proximal portion or section (also referred to herein as a first angled portion) 176. An angle of the distal portion 174 in comparison with a vertical reference line is different than an angle of the proximal portion 176 in comparison with the same vertical reference line. More specifically, the distal portion 174 has a more vertical orientation in comparison with the proximal portion 176. In some embodiments, the angle of the distal portion 174 may be less than 5 degrees, less than 4 degrees, less than 3 degrees, or less than 2 degrees in comparison with the vertical reference line while the angle of the proximal portion 176 is greater than 3 degrees, greater than 4 degrees, greater than 5 degrees, or greater than 6 degrees in comparison with the vertical reference line. The angle of the distal portion 174 is typically greater than 1 degree while the angle of the proximal portion 176 is typically less than 15 degrees or 10 degrees. In the illustrated embodiment, the distal portion 174 has an angle or approximately 2.5 degrees in comparison with the vertical reference line while the proximal portion 176 has an angle of approximately 6 degrees. It should be noted that the values illustrated in FIG. 3B are for demonstrative purposes only and may be varied as desired or required for a given application. Thus, the illustrated values do not limit the scope of the description or claims in any manner.

The distal portion 174 is typically smaller lengthwise than the proximal portion 176 and, in some embodiments, may extend less than 40% of the length of the tooth 170, less than 35% of the length of the tooth 170, or less than 30% of the length of the tooth 170. The proximal portion 176 may extend along a remainder of the length of the tooth 170 as desired. In one embodiment, only the teeth 147 of the pawl disc 140 may have a varied angle construction as illustrated in FIG. 3B. Alternatively, in other embodiments, only the teeth 126 of the spool 120 may have a varied angle construction as illustrated in FIG. 3B. In other embodiments, both the teeth 147 of the pawl disc 140 and the teeth 126 of the spool 120 may have a varied angle construction as illustrated in FIG. 3B.

The latter embodiment in which both teeth, 126 and 147, have a varied angle construction may be preferred because the more vertically oriented tips, or distal ends, of each tooth greatly enhance reengagement or remeshing of the teeth, 126 and 147. The more vertically oriented surfaces of each tooth, 126 and 147, further reduces the force that is required to move the pawl disc 140 axially upward and out of engagement with the spool 120 and reduces wear on the faces and tips of each tooth, 126 and 147. These advantages allow the size of the teeth, 126 and 147, to be reduced, which may reduce the overall size or height of the reel based closure device 100. The increased angle of the proximal portion of each tooth, 126 and 147, facilitates in maintaining an engagement of the pawl disc 140 and spool 120, especially as tensile forces in the tension member are increased. This increased engagement of the pawl disc 140 and spool 120 minimizes accidental disengagement of the two components, which would cause unwanted loosening of the tension member.

FIG. 3C illustrates the teeth 126 of the spool 120 having the varied angle construction described above. Specifically, each tooth 126 includes a proximal portion, or first angled portion, 127a and a distal portion, or second angled portion, 127b in which the distal portion 127b has a more vertical orientation than the proximal portion 127a in comparison with a vertical reference line or plane. In some instance, the proximal portion 127a may directly abut or contact the distal portion 127b while in other instances, a transition portion or zone may be formed between the proximal portion 127a and the distal portion 127b, as illustrated by the area between the shaded proximal portion 127a and distal portion 127b in FIG. 3C.

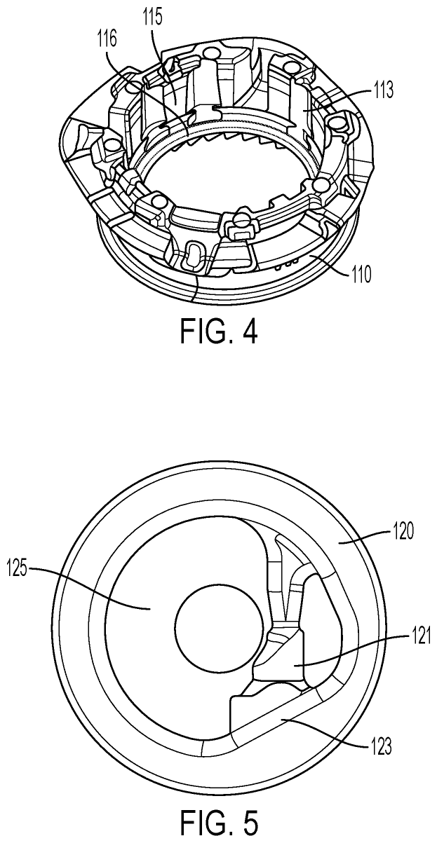

FIG. 4 illustrates a bottom perspective view of the housing 110. The bottom perspective view shows the interior of the lower portion of housing 110 that is positioned axially below the inner annular ridge 116. As illustrated, the lower portion of the housing 110 includes a plurality of channels, recesses, or depressions 113 (hereinafter channels 113) that are formed in the inner surface of the housing wall and that extend axially along the housing wall. In some embodiments, the channels 113 may extend from the inner annular ridge 116 to a bottom edge or surface of the housing 110, which may be open. In other embodiments, the channels 113 may extend along only a portion of the housing 110, but not up to the bottom edge/surface and/or to the inner annular ridge 116. In some embodiments, the housing 110 may include between 4 and 20 channels 113, more commonly between 5 and 12 channels 113, and most commonly between 6 and 10 channels 113. In a specific embodiment, the housing 110 may include 8 or 9 channels 113.

The channels 113 define inward extending surface 115 that are positioned between adjacent channels 113. The inward extending surface 115 extend axially along the housing wall and may extend fully or partially from the inner annular ridge 116 to a bottom edge or surface of the housing 110, which may be open. The formation of the channels 113 on the inner surface of the housing wall reduces frictional contact between the tension member and housing 110. Specifically, the use of the channels 113 results in the tension member (not shown) contacting one or more surfaces 115 of the housing 110 as the spool 120 is rotated in the tightening or loosening direction within the housing's interior. The upper and lower flanges of the spool 120 may also briefly contact or more surfaces 115 of the housing wall as the spool 120 is rotated within the housing's interior. Since the channels 113 are recessed into the inner wall of the housing 110, the tension member, or spool's flanges, do not contact the material in the channels 113 and instead only contact the material on a surface 115, or surfaces, of the housing 110. In this manner, contact between the tension member, or spool's flanges, and the inner wall of the housing 110 is reduced, which reduces a friction or drag that is induced on the tension member, or spool's flanges. The reduced friction or drag results in an increase in tension induced in the tension member as the tension member is wound about the spool 120 since more of the input energy is directly transferred to the tension member. Similarly, the reduced friction or drag results in the tension member being more easily unwound from the spool 120 during loosening of the tension member.

FIG. 5 illustrates a cross-sectional top or bottom view of the spool 120. The spool 120 includes a central cylindrical body 125 around which the tension member is wound and unwound during tensioning and loosening of the tension member. The spool 120 also includes a channel or recess 121 that extends into or is defined in the central cylindrical body 125 of the spool. The channel or recess 121 (hereinafter channel 121) typically extends through the central cylindrical body 125 from one side of central cylindrical body 125 to an opposite side of the central cylindrical body 125. The channel 121 is positioned on a single side of a central aperture of the spool 120. The channel 121 is used in attaching the tension member to the spool as further described in U.S. Patent Application No. 62/101,283, filed Jan. 8, 2015, entitled “Closure System Components for Enabling Easy Attachment of Lace”, the entire disclosure of which is incorporated by reference herein.

The spool 120 commonly includes a single channel 121, although in some embodiments the spool 120 may include another channel 121 positioned on an opposite side of the central aperture. As further illustrated in FIG. 5, the cylindrical body 125 of the spool 120 is not perfectly cylindrical. Rather, the cylindrical body 125 has a more oval profile when viewed in cross section from a top or bottom view as in FIG. 5. The more oval profile of the cylindrical body 125 results in the cylindrical body 125 of the spool 120 having an asymmetrical shape. This shape reduces wear and/or pressure points on the tension member, thereby reducing a failure of the tension member after prolonged use.

Additional details of components of the reel based closure device 100 are described in U.S. Patent Application No. 62/101,283, incorporated by reference herein. The various components may also be used with the reel based closure device described in U.S. patent application Ser. No. 16/864,849, filed May 1, 2020, entitled “Reel Based Closure System”, the entire disclosure of which is incorporated by reference herein.

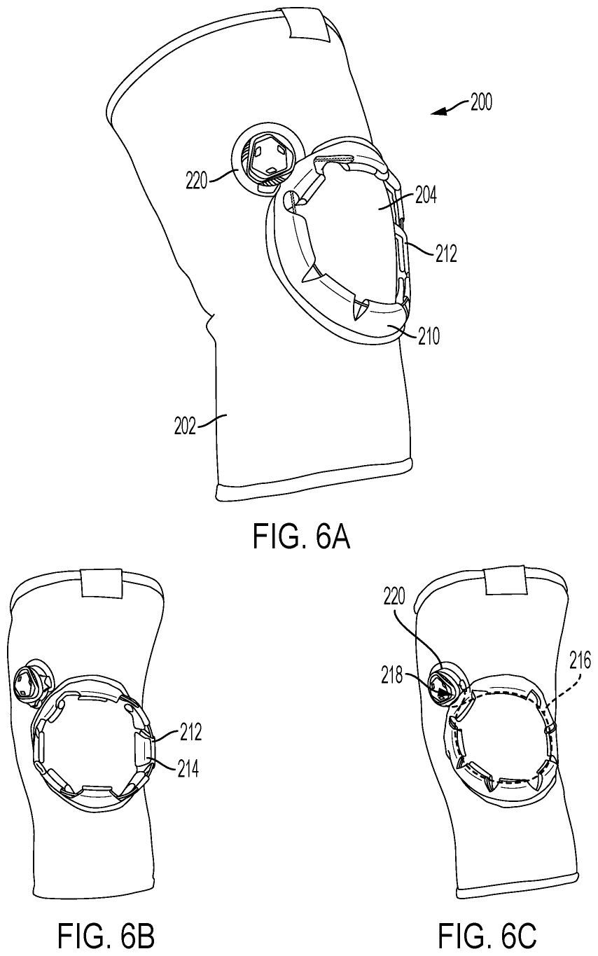

The embodiments of FIGS. 6A-12G relate to orthotic braces that are positionable about a limb in order to support the limb and promote healing and/or provide support and reinforcement to the limb. In a specific embodiment, the brace is a knee brace that is positionable about the leg and that is configured to support the patella of the knee. The embodiments described herein centrally hold the patella with the use of a single tightening mechanism. For ease in describing the embodiments herein, the orthotic brace will be generally referred to as a knee brace.

In addition to the reel based closure device described herein, other tightening mechanism that may be used include pull cord systems, motorized system or device, conventional lacing, or any other systems that is able to tension a lace or cord. Exemplary embodiments of such devices are further described in U.S. patent application Ser. No. 13/098,276, filed Apr. 29, 2011, titled “Reel Based Lacing System”, U.S. patent application Ser. No. 14/328,521, filed Jul. 10, 2014, titled “Closure Devices Including Incremental Release Mechanisms and Methods Therefor,” and U.S. patent application Ser. No. 12/623,362, filed Nov. 20, 2009, titled “Reel Based Lacing System”, the entire disclosures of which are incorporated by reference herein.

In some embodiments, the knee brace includes a petal configuration that is positionable around the patella. The petal configuration may include a plurality of wedge shaped petals that are tensionable in order to pull the petals into contact with the knee around the patella and thereby support the patella and knee. FIGS. 6A-C illustrate a first embodiment of a knee brace 200 that includes a petal configuration. The knee brace 200 includes a brace body 202 and a support member 210 that is attached to the brace body 202 via stitching, adhesive bonding, insert molding, and the like. When attached to the brace body 202, the support member 210 forms or defines an opening 204 that at least partially surrounds the patella. In the illustrated embodiment, the support member 210 is a single annular member that completely surrounds the patella without any breaks or discontinuities in the support member 210. In other embodiments, the support member may be formed of two or more pieces that are arranged on the brace body 202 so that the support member 210 surrounds the patella.

The support member 210 includes a plurality of petals or fingers 212 (hereinafter petals 212) that each protrude radially inward toward a center of the knee or patella. The petals 212 may be wedge shaped components and may be padded on an underside of the petals 212 that contacts the knee so as to comfort and cushion the knee. A small slit or opening may be formed between adjacent petals 212, which may allow, or aid in, the plurality of petals 212 constricting inward about the knee and patella. The petals 212 may have foam wedge padding within an interior of the petals, which may focus pressure around the patella in a comfortable manner.

A guide member 214 is operably coupled with each of the petals 212. An example of a guide member 214 is illustrated in FIG. 6B. In the illustrated embodiments, the guide member 214 is positioned on an interior side of each petal 212. In other embodiments, the guide member 214 may be positioned on an exterior of each petal, or the guide member may be integrated into the petal 212. For example, one or more petals 212 may be looped inward to form a channel through which the tension member is inserted or positioned. Each of the petals 212 may have the same guide member configuration, or the guide members may be arranged differently on each of the petals 212. For example, one or more petals 212 may have interior facing guide members 214, one or more petals 212 may have one or more exterior facing guide members 214, and/or one or more petals may have an integrated guide member 214.

A reel based closure device 220 is also attached to the brace body 202 adjacent to the support member 210. In some embodiments, the reel based closure device 220 is positioned on the brace body 202 so that the reel based closure device 220 is vertically above the support member 210, although the reel based closure device 220 may be positioned elsewhere as desired. The reel based closure device 220 may be positioned in a horizontal center of the support member 210 or may be positioned offset from the horizontal center as illustrated in FIGS. 6A-C. In some embodiments, the reel based closure device 220 may be positioned offset from the horizontal center so that the reel based closure device 220 is closer to the inner portions of the leg, which may protect the reel based closure device 220 from contact with external objects that could cause unwanted damage to the reel based closure device 220 and/or cause unwanted opening of the reel based closure device 220 and loosening of the tension member.

A tension member or lace is operably coupled with the reel based closure device 220 so that an operation of the reel based closure device 220 (e.g., rotation of the knob) effects tensioning of the tension member. As mentioned herein, the tension member (not shown) is operably coupled with each of the guide members 214, such as by inserting the tension member through an opening of each guide member, so that tensioning of the tension member causes the petals 212 to constrict inward around or about the patella. The lace path of the tension member is illustrated by the dashed line 216 in FIG. 6C. The lace path of the tension member forms or defines a loop or circle around the patella. Tensioning of the tension member constricts or decreases a diameter of the loop or circle, which pulls the petals 212 inward toward a center of the knee. This action causes the petals to exert an inward and downward pressure on the leg and knee around the patella, which supports and reinforces the patella. The petals 212 may be arranged so that they contact the patella and/or contact the tissue immediately surrounding the patella.

As illustrated in FIG. 6C, the lace path of the tension member may be arranged so that the tension member crosses itself near the reel based closure device 220. In other embodiments, the lace path may not cross itself along an entire length of the lace path. Crossing of the lace path as illustrated in FIG. 6C introduces friction into the lacing system, which may inhibit shifting of the tension member within the guide members 214 and thereby maintain a fit and hold of the support member 210 about the patella.

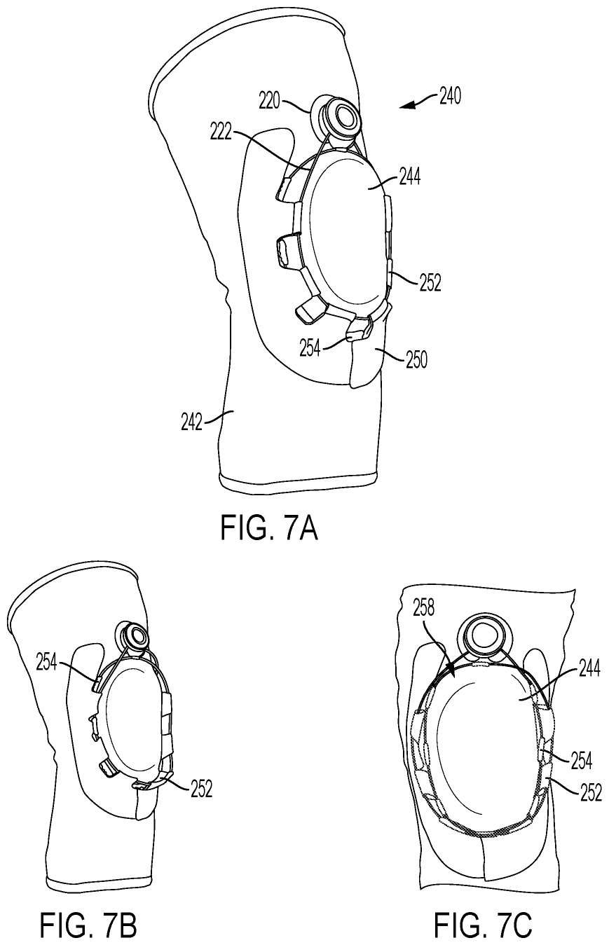

FIGS. 7A-C illustrate a second embodiment of a knee brace 240 that includes a petal configuration. The knee brace 240 includes a brace body 242 and a support member 250 that is attached to the brace body 242 via stitching, adhesive bonding, insert molding, and the like. In the illustrated embodiment, the support member 250 is a U-shaped member that partially surrounds the patella. The U-shaped support member 250 may be formed of a single continuous member or a plurality of pieces that are arranged on the brace body 242 and attached thereto to form the U-shaped support member 250. In other instances, the support member 250 may completely surround the patella without any breaks or discontinuities in the support member 250.

The support member 250 partially or completely surrounds a support pad 244 that is positioned atop the patella. In a specific embodiment, the support pad 244 is a gel donut-shaped padding that surrounds the patella and the lateral and medial sides of the knee. The support member 250 partially or completely surrounds the support pad 244. The support member 250 includes a plurality or set of overlapping petals or fingers (hereinafter petals) that provide a canopy effect atop the support pad 244. The support member 250 may be particularly useful for knee braces that include donut shaped support pads 244. Specifically, the support member 250 includes a first set of petals 254 that are positioned at a base of the support pad 244 and a second set of petals 252 that are positioned atop support pad 244. Each petal of the first set 254 and second set 252 is configured so that a tension member 222 may be guided or routed along a path by the respective petals. The first set of petals 254 and the second set of petals 252 may include guides that are positioned on an interior portion of the petals, an exterior portion of the petals, and/or are that integrated with the respective petals as previously described. The tension member 222 may be inserted through the respective guides of the petals to allow the petals to route or guide the tension member 222 along the lace path.

Each of the petals of the first set 254 and the second set 252 protrude radially inward toward a center of the patella or knee. The petals of the second set 252 protrude farther radially inward than the petals of the first set 254. This first set of petals 254 and the second set of petals 252 produces an inward and downward pressure on the support pad 244, which in turns produces an inward and downward pressure on the patella and knee. The petals of the first set 254 and second set 252 may be wedge shaped components and may be padded on an underside of the petals as desired. A small slit or opening may be formed between adjacent petals of the first set 254 and second set 252, which allows, or aids in, the plurality of petals constricting inward about the support pad 244.

A reel based closure device 220 is also attached to the brace body 242 adjacent to the support member 250. In some embodiments, the reel based closure device 220 is positioned on the brace body 242 so that the reel based closure device 220 is vertically above the support member 250 and between opposing ends of the U-shaped support member 250. The reel based closure device 220 may be positioned elsewhere as desired. The reel based closure device 220 may be positioned in a horizontal center of the support member 250 as illustrated.

The tension member 222 is operably coupled with the reel based closure device 220 so that an operation of the reel based closure device 220 (e.g., rotation of the knob) effects tensioning of the tension member. The tension member 222 is operably coupled with each of the petals so that one continuous path is formed by the tension member 222. Specifically, starting from the reel based closure device 220 and following the lace path in a counterclockwise direction, the tension member is routed from the reel based closure device 220 and through each of the guides of the second set of petals 252. The tension member 222 is then routed from an upper right hand petal of the second set 252, over a top portion 258 of the support pad 244, and through each of the guides of the first set of petals 254. The tension member 222 is routed from an upper right hand petal of the first set 254 and to the reel based closure device 220. In this manner the tension member 222 completely encircles the support pad 244. Tensioning of the tension member 222 causes the first set of petals 254 to constrict radially inward, which produces a mostly inward pressure on the support pad 244. Tensioning of the tension member 222 also causes the second set of petals 252 to constrict radially inward, which produces an inward pressure and a greater downward pressure on the support pad 244. The support member 250 supports and reinforces the patella. The support member 250 also allows for dual level adjustment with a single reel based closure device 220.

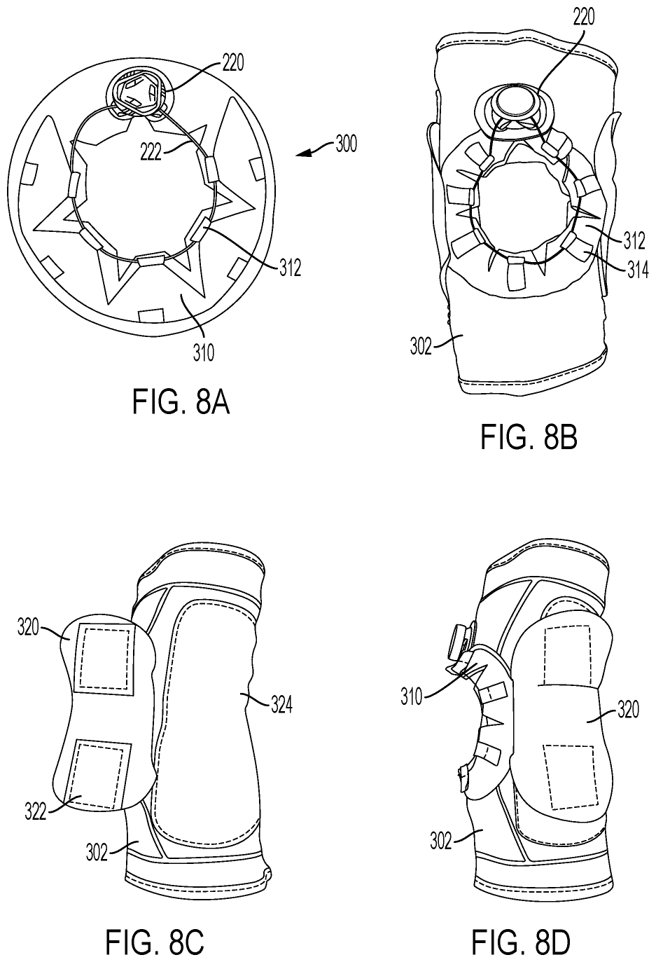

FIGS. 8A-D illustrate a third embodiment of a knee brace 300 that includes a petal configuration. The knee brace 300 includes a brace body 302 and a support member 310 that is attached to the brace body 302 via stitching, adhesive bonding, insert molding, and the like. In the illustrated embodiment, the support member 310 completely encircles or surrounds the patella, although in other embodiments, the support member 310 may only partially surround the patella. The support member 310 may be formed of a single continuous member or a plurality of pieces that are arranged on the brace body 302 and attached thereto.

As with the previous embodiment, the support member 310 may partially or completely surrounds a support pad (not shown) that is positioned atop the patella. The support pad may be a gel donut-shaped padding, or any other padded member, which surrounds the patella and the lateral and medial sides of the knee. In some instance, the knee brace 300 may not include a support pad and the support member 310 may more directly engage the patella, or other portions of the leg or knee, so as to comfort and cushion the knee.

The support member 310 includes a plurality of petals or fingers 312 (hereinafter petals) that provide a canopy effect atop the support pad or patella. An opening is formed between adjacent petals 312, which allows or aids the petals 312 to constrict inward about the support pad or patella. The petals 312 may include interior padding, which may focus pressure around the patella in a comfortable manner. Each petal 312 is configured so that the tension member 222 may be guided or routed along a path by the respective petals. The petals 312 include guides that are positioned on an interior portion of the petals, an exterior portion of the petals, and/or are that integrated with the respective petals as previously described. The tension member 222 is inserted through the respective guides of the petals 312 to allow the petals to route or guide the tension member 222 along the lace path. When the tension member 222 is tensioned via operation of the reel based closure device 220 (e.g., rotation of the knob), the petals 312 constrict inward about the patella, or a support pad, as previously described.

The reel based closure device 220 may be positioned at an uppermost position or location of the lace path. In such instances, the tension member 222 is immediately routed from the reel based closure device 220 to adjacent petals 312 that are positioned on opposing sides of the reel based closure device 220. The tension member 222 may or may not cross itself near the reel based closure device 220 depending on whether it is desired to introduce an amount of friction in the system. When the tension member 222 does not cross itself, the petals 312 may more easily adapt to movement of the knee, such as due to bending and flexing of the leg.

In some embodiments, one or more panels or straps 320 (hereinafter panel 320) may be attached to the support member 310. The panel, or panels, 320 are typically attached to one or more sides of the support member 310 and are operable to introduce a desired force or pressure on the knee or patella. For example, in the illustrated embodiment, the knee brace 300 includes two panels 320 that are attached to opposing sides of the support member 310. More specifically, a front edge of each panel 320 is attached to the support member 310 via stitching, adhesive bonding, molding, and the like. A main body of each panel 320 is moveable about the knee brace 300. For example, the front edge of each panel 320 may be the only non-removable attachment of the panel 320 to the knee brace 300 and support member 310. This coupling allows the panel 320 to pivot about the attachment of the front edge and to be pulled laterally or rearward about the knee brace 300.

Each panel 320 also includes a fastener 322 that enables the panels 320 to be attached to a main body of the knee brace 300. In the illustrated embodiment, the panel 320 includes a hook or loop fastener 322 that is configured to fasten to a hook or loop fastener 324 that is positioned on the main body of the knee brace 300. In other embodiments, the panel 320 may include other fasteners, such as magnetic fasteners, buckles, snaps, clamps, and the like. The panels 320 are designed to be pulled rearward relative to the knee brace 300 and attached to the main body of the knee brace. This action imparts a lateral force on the support member 310 (i.e., in a circumferential direction around the knee/leg), which pulls the support member 310 into increased contact with the patella, thereby providing increased support to the patella and knee. When the knee brace 300 includes a pair of panels 320 as illustrated in FIGS. 8C-D, the pair of panels 320 may impart an equal and oppositely directed force or pressure on the patella. The use of the panels 320 as illustrated in FIGS. 8C-D may increase the lateral support on the patella in comparison with the support provided by the use of the support member 310 alone. The force or pressure imparted by the pair of panels 320 illustrated in FIGS. 8C-D may be directed behind the leg and/or downward around the knee.

It should be noted that while a pair of panels 320 is illustrated as being used in FIGS. 8C-D, in other instances, the knee brace 300 may include a single panel 320 and/or the panel may be positioned elsewhere as desired. For example, the knee brace 300 may include a panel that is positioned below the support member 310 and that is configured to impart a downward force on the support member 310 directed toward the foot. Similarly, the knee brace 300 may include a panel 320 that is positioned above the support member 310 and that imparts un upward force on the support member 310 directed toward the upper leg. In some embodiments, the knee brace may include a plurality of panels 320 that surround the support member 310 and that are independently operable to impart a force on the support member 310 as desired. In this manner, the force or pressure that is exerted on the patella and knee may be varied and/or customized as desired.

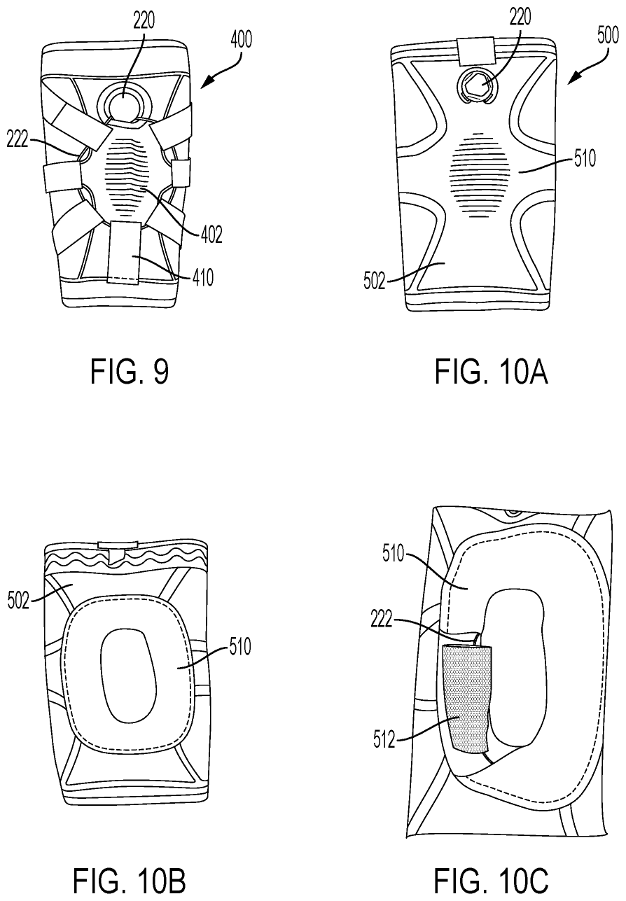

FIG. 9 illustrates a fourth embodiment of a knee brace 400 that includes a patella or knee support configuration. The knee brace 400 includes a brace body 402 and a plurality of straps 410 that are coupled with the brace body 402 and that are arranged to function in a manner similar to the support members previously described. Each of the straps 410 has a proximal end that is attached to the brace body 402 via stitching, adhesive bonding, insert molding, and the like. The straps 410 are arranged so that a distal end of the strap is oriented or directed toward the patella or knee. The straps 410 are further arranged so that the distal ends of each of the straps 410 encircles the patella in a circular or oval fashion. Each of the straps 410 may be oriented at an angle relative to the adjacent straps 410.

A lace guide is formed in, or attached to, the distal end of each strap. The lace guide is configured to guide or direct the tension member 222 about a path that encircles the patella as previously described. For example, the distal end of each strap 410 may be folded backward to form a loop within which the tension member 222 is inserted. In other embodiments, a separate guide, such as a plastic guide component, may be attached to the distal end of one or more straps 410. An advantage of forming loops in the distal ends of the straps 410 is that the straps 410 are flexible and able to adapt to the shape of the leg and knee, thereby eliminating or reducing pressure points.

The tension member 222 is inserted through each of the straps and is routed from the reel based closure device 220, which may be positioned at an uppermost point of the lace path, or elsewhere about the knee brace 300 as desired. Tensioning of the tension member 222 via operation of the reel based closure device 220, pulls each of the straps 410 inward relative to the knee, which creates an inward and slightly downward pressure on the patella and knee. This pressure may support the patella and/or provide increased cushioning and comfort to the knee. The use of the straps 410 may create a “wrapping” force about the knee that begins at the proximal end of the respective straps 410 and that wraps around the knee toward the patella. In some embodiments, one or more straps 410, or each of the straps 410, may include padding or cushioning on an inner surface that increases comfort and/or provides pressure in a desired position about the leg and knee. The number and positioning of the straps 410 may be varied to impart a desired force or pressure on the leg and knee. Similarly, the length and width of the straps 410 may be varied to induce a desired force or pressure on the leg and knee.

FIGS. 10A-C illustrate a fifth embodiment of a knee brace 500 that includes a support member 510 for supporting and/or cushioning the patella and knee. The knee brace 500 includes a brace body 502 and a reel based closure device 220. The reel based closure device 220 is attached to the brace body 502 and is positioned on an exterior surface of the brace body 502. The reel based closure device 220 may be positioned at an uppermost point of a lace path as previously described, or elsewhere on the brace body 502 and knee brace 500 as desired.

Referring to FIGS. 10B-C, illustrated is a view of an interior of the knee brace 500, such as by inverting the knee brace so that it is inside out. In contrast to the previous embodiments, the support member 510 is positioned on an interior of the knee brace 500. As such, the support member 510 and reel based closure device 220 are positioned on opposite surfaces of the knee brace 500. The support member 510 is positioned on the interior of the knee brace 500 to partially or fully surround the patella. The reel based closure device 220 is operably coupled with a tension member 222, which is tensionable to constrict the support member 510 about the patella and thereby support and/or cushion the patella and knee. The tension member 222 is routed in an interior of the support member 510 so that it does not contact the skin of the wearer and is not visible from an exterior of the knee brace 500. The tension member may be coupled with one or more cushion members or padding 512 (hereinafter padding 512).

In some embodiments, the padding 512 includes or functions as a guide that routes or directs the tension member 222 about the patella. In other embodiments, the tension member 222 may be attached to one or more padding 512 pieces so that tensioning of the tension member 222 causes the padding 512 to move or slide within the support member 510 and constrict about the patella. In a specific embodiment, the padding 512 includes two padding pieces that are wedged shaped and configured to slide atop one another. As the wedges slide atop one another, an increased downward force is applied to the patella or knee due to an increased volume of material within the support member 510. For example, the padding 512 may include two U-shaped wedges that overlap with one U-shaped wedge piece facing upward and one U-shaped wedge piece facing downward. As the oppositely facing U-shaped wedge pieces slide atop one another, a downward pressure is created around the patella and knee.

As the tension member 222 is tensioned via operation of the reel based closure device 220, the support member 510 constricts inward, thereby imparting an inward and slightly downward pressure on the patella and knee, which supports and/or cushions the patella and knee. The knee brace 500 of FIGS. 10A-C may enable a more direct force to be imposed on the patella since the support member 510 is positionable directly atop the knee and does not need to press downward and inward on exterior material layers of the knee brace 500. The knee brace 500 of FIGS. 10A-C may also offer a more desirable visual appeal since the support member 510 and tension member 222 are concealed from external view.

Additional patella support concepts are illustrated in the Appendix of the drawings. Each of these systems includes a reel based closure device and tension member that are operable to impart a supporting and/or cushioning force on the patella and knee. The arrangement and orientation of the various components is readily evident upon visual examination of the various embodiments of the appendix, as is the operation and function of the support member or patella supporting components. Applicant reserves the right to describe the features of the various embodiments in a future application and it is recognized that a description of features that are readily apparent upon visual examination of the embodiments does not constitute new matter.

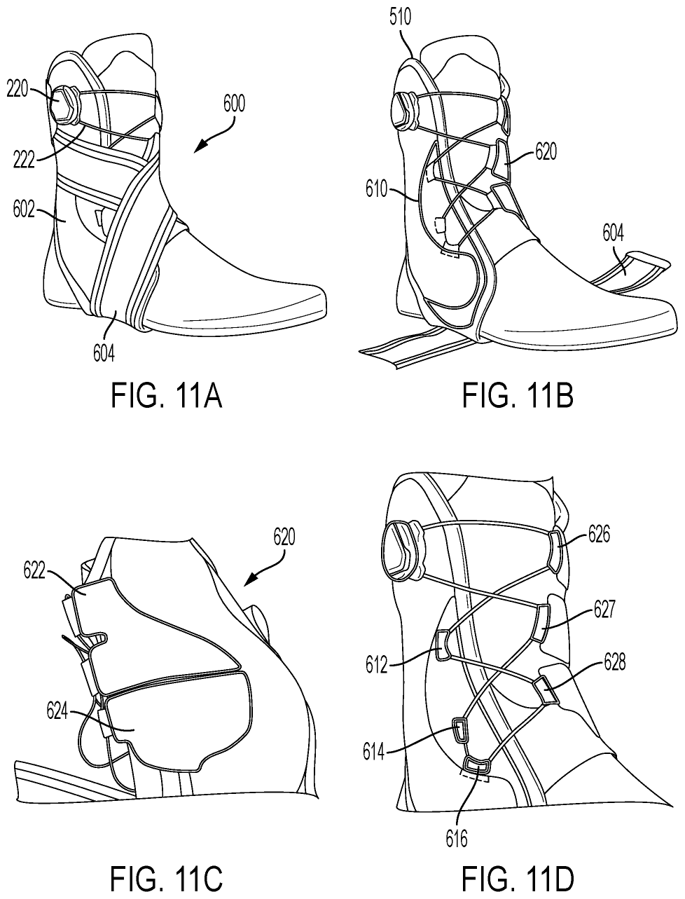

Referring to FIGS. 11A-D, illustrated is an embodiment of an ankle brace 600 that may be fit about the ankle to support and strengthen the ankle. The ankle brace 600 includes a brace body 602 having a lateral side 610 and a medial side 620 that are designed for positioning on opposing sides of a wearer's foot. The lateral side 610 and medial side 620 are typically attached along a rear portion that is positioned behind the wearer's leg and along a lower portion that is positioned below the wearer's foot. The lateral side 610 and medial side 620 are typically not attached along a front portion, which allows the ankle brace 600 to be opened and fit about the wearer's foot.

The ankle brace 600 also includes a primary closure system and a secondary closure system that are designed to close the ankle brace 600 about the foot and secure the ankle brace to the foot in a comfortable and supportive manner. The primary closure system comprises or consists of a reel based closure device 220, a tension member 222, and a plurality of guides that guide or direct the tension member 222 about a path along the ankle brace 600. These components function in a manner as described herein. The reel based closure device 220 may be positioned near an upper portion or cuff of the ankle brace as illustrated in FIGS. 11A-D, or may be positioned elsewhere about the ankle brace 600 as desired.

The secondary closure system consists of one or more straps 604 that may be wrapped and secured about the ankle brace 600 by the wearer. The primary closure system may provide a first type of closure, such as a gross closure and fit of the ankle brace 600 about the lower leg of the wearer, while the secondary closure system provides a second type of closure, such as a secure fit of the ankle brace 600 about the foot.

FIG. 11B illustrates the strap 604 uncoupled from the brace body 602 so that the lateral side 610 and medial side 620 of the brace body 602 are readily apparent. The lateral side 610 includes a stabilizer (shown in outline) that stabilizes the lateral side 610, and more specifically an edge of the lateral side, about the wearer's foot. The stabilizer reduces buckling of the lateral side 610 as the ankle brace 600 is closed and tightened about the foot and as the ankle brace 600 is worn. The stabilizer may include multiple layers of material, such as one or more layers of looped fabric and a woven polymer (TPU) backing. As illustrated in FIG. 11C, the medial side 620 also includes a stabilizer (shown in outline) that stabilizes the medial side 620 of the brace body 602 about the wearer's foot. In a specific embodiment, the medial side 620 includes a pair of stabilizers as illustrated in FIG. 11C. The stabilizers on the medial side 620 are separate or independent panels or members and are movable relative to one another to a degree. The use of separate and movable stabilizer members enables better conformance and fit to be achieved around the mid foot. The stabilizers of the medial side 620 may include multiple material layers, such as one or more layers of looped fabric and a woven polymer (TPU) backing.

The stabilizers of the lateral side 610 and/or medial side 620 may be shaped to accommodate the shape and size of the wearer's foot. The stabilizers provide a mostly static support system while offering a degree of conformance or give. As illustrated in FIG. 11D, one or more guides are permanently or removably coupled with the stabilizers of the lateral side 610 and/or medial side 620. The lateral side 610 includes an upper guide 612 and a pair of lower guides, 614 and 616. The upper guide 612 routes or directs the tension member 222 between an upper guide 626 and a mid-guide 627 on the medial side while the lower guides, 614 and 616, function in tandem to route or direct the tension member 222 between the mid-guide 627 and a lower guide 628 on the medial side. The use of the lower guides, 614 and 616, that function in tandem eases the tension in the tension member 222 as the tension member is routed 180 degrees or more and reduces buckling of the bottom portion of the lateral side 610 of the brace body 602. The upper guide 626 and mid-guide 627 are attached to an upper stabilizer on the medial side 620 while the lower guide 628 is attached to a lower stabilizer on the medial side 620.

Tensioning of the tension member 222 causes the lateral side 610 and medial side 620 to be pulled toward one another, which constricts the ankle brace 600 about the wearer's ankle. The straps 604 may then be wrapped about the brace body 602 and fastened together according to the wearer's preference. The straps 604 may be fastened via hook and loop fasteners, clamps, buckles, snaps, magnetic fastening, or using any other means of mechanism fastening.

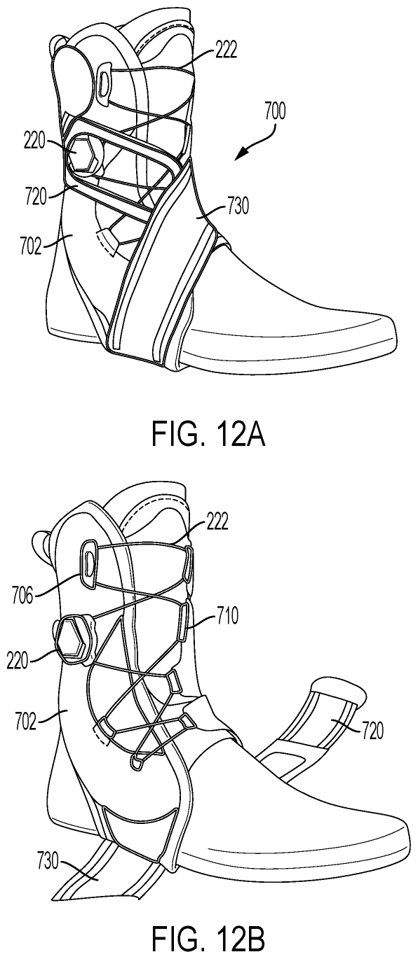

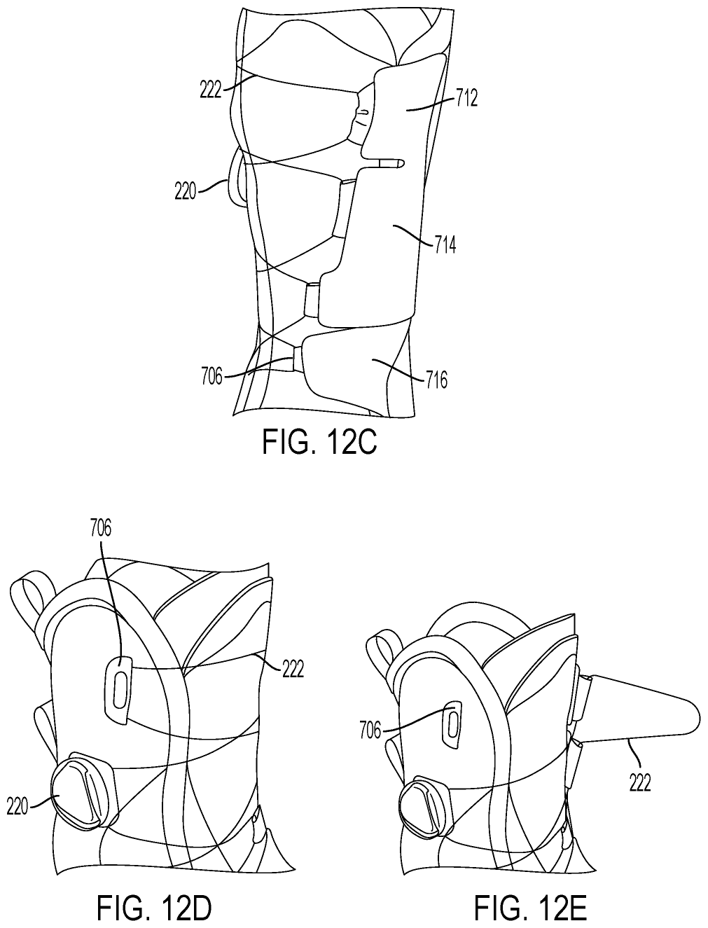

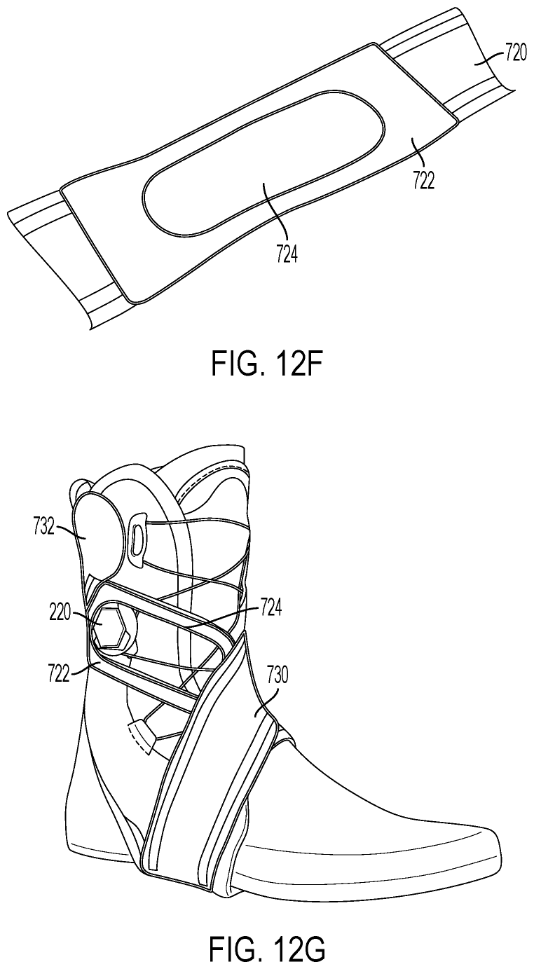

FIGS. 12A-G illustrate another embodiment of an ankle brace 700 that may be fit about the ankle to support and strengthen the ankle. The ankle brace 700 includes a brace body 702 having a lateral side and a medial side that are designed for positioning on opposing sides of a wearer's foot. The lateral side and medial side are typically attached along a rear portion that is positioned behind the wearer's leg and along a lower portion that is positioned below the wearer's foot. The lateral side and medial side are typically not attached along a front portion, which allows the ankle brace 700 to be opened and fit about the wearer's foot.

The ankle brace 700 includes a primary closure system (e.g., reel based closure device 220, tension member 222, and guides) and a secondary closure system (e.g., one or more straps) that are designed to close the ankle brace 700 about the foot and secure the ankle brace to the foot in a comfortable and supportive manner. The reel based closure device 220 may be positioned near an upper portion or cuff of the ankle brace or may be positioned elsewhere about the ankle brace 700 as desired. In a specific embodiment, the reel based closure device 220 is positioned near a mid-point of the lateral side, or near a mid-point of the lace path of the tension member 222, as illustrated in FIG. 12B. Placement of the reel based closure device 220 in this manner may offer a more uniform fit and reduce unwanted shifting or movement of the tension member 222 about the lace path. Placement of the reel based closure device 220 on the mid-point of the lace path is enabled by the use of a dial strap 720 as explained herein below. The primary closure system may provide a first type of closure, such as a gross closure and fit of the ankle brace 700 about the lower leg of the wearer, while the secondary closure system provides a second type of closure, such as a secure fit of the ankle brace 700 about the foot.

FIG. 12B illustrates a pair of straps, 720 and 730, uncoupled from the brace body 702 so that the lateral side and medial side of the brace body 702 are more readily apparent. The lateral side includes a stabilizer (shown in outline) that stabilizes the lateral side, and more specifically an edge of the lateral side, about the wearer's foot. The stabilizer reduces buckling of the lateral side as the ankle brace 700 is closed and tightened about the foot and as the ankle brace 700 is worn. The stabilizer may include multiple layers of material, such as one or more layers of looped fabric and a woven polymer (TPU) backing.

As illustrated in FIG. 12C, the medial side also includes a stabilizer (shown in outline) that stabilizes the medial side of the brace body 702 about the wearer's foot. In a specific embodiment, the medial side includes a pair of stabilizers as illustrated in FIG. 12C. The stabilizers on the medial side are separate or independent panels or members and are movable relative to one another to a degree. The use of separate and movable stabilizer members enables better conformance and fit to be achieved around the mid foot. The stabilizers of the medial side may include multiple material layers, such as one or more layers of looped fabric and a woven polymer (TPU) backing.

The stabilizers of the lateral side and/or medial side may be shaped to accommodate the shape and size of the wearer's foot. The stabilizers provide a mostly static support system while offering a degree of conformance or give. As illustrated in FIGS. 12B-C, one or more guides are permanently or removably coupled with the stabilizers of the lateral side and/or medial side. In the illustrated embodiment, the lateral side includes three guides 706 with one guide positioned above the reel based closure device 220 and two guides positioned below the reel based closure device 220. The medial side include four guides 710, in which the three upper guides 710 are attached to an upper stabilizer and in which the lowest guide 710 is attached to a lower stabilizer. The three lateral guide 706 and four medial guide 710 arrangement enables an even and uniform fit to be achieved.

In some embodiments, the upper stabilizer may be segmented into an upper portion 712 and a lower portion 714, which are slightly separated by a groove or channel between the two portions. The segmenting of the upper guide into upper and lower portions, 712 and 714, enables the upper stabilizer to conform and adjust to the shape of the wearer's leg to a greater degree. The lower stabilizer 716 is separate from the upper stabilizer and is able to move, shift, and adjust independent of the upper stabilizer, which allows better conformity and fit, especially around the mid-foot. The material of the lower stabilizer 716 may be tapered on an underside to reduce pressure on the edge of the panel.

As illustrated in FIG. 12D-E, in some embodiments, the upper lateral guide 706 may be a releasable guide, which means that the tension member 222 may be detached from the guide 706. The guide 706 includes an open back end that enables the tension member 222 to be quickly inserted and removed from a channel defined in the guide 706. Removal and insertion of the tension member 222 within the channel of the guide 706 allows the tension in the ankle brace 700 to be quickly adjusted, which facilitates in donning and doffing of the ankle brace 700 about the wearer's foot. FIG. 12D illustrates the tension member 222 positioned within the channel of the guide 706 while FIG. 12E illustrates the tension member 222 removed from the guide 706.

The dial strap 720 is illustrated in FIG. 12F. The dial strap 720 includes a mid-20 section or body 722 having a window or elongate channel 724 (hereinafter elongate channel 724) that is designed to accommodate the reel based closure device 220. Specifically, the elongate channel 724 has a width that is slightly wider than a width of the reel based closure device 220, which allows the dial strap 720 to be positioned over the reel based closure device 220 with the reel based closure device 220 positioned in the elongate channel 724 of the dial strap 720. The elongate channel 724 also has a longitudinal length that allows the dial strap 720 to slide or move longitudinally while the reel based closure device 220 is positioned within the elongate channel 724. In this manner, the position of the reel based closure device 220 on the ankle brace 700 does not interfere with the positioning and/or closure of the dial strap 720 about the ankle brace 700. The use of the elongate channel 724 in the dial strap 720 also enables the reel based closure device 220 to be positioned on or near a mid-point of the lace path without negatively affecting the positioning and closure of the dial strap 720 about the ankle brace 700. In some embodiments, the body 722 of the dial strap 720 is reinforced in comparison with a remainder of the dial strap 720, which reduces or prevents breakage of the dial strap 720 at or near the elongate channel 724. A reinforcing material or layer may be coupled with the dial strap 720 at or near the elongate channel 724 to reinforce the body 722.

FIG. 12G illustrates the dial strap 720 wrapped about the ankle brace 700 and fitted or positioned over the reel based closure device 220 so that the reel based closure device 220 is positioned in the elongate channel 724 of the dial strap's body 722. FIG. 12G also illustrates that a secondary strap 730 is wrapped about the ankle brace 700 and over the dial strap 720. A distal end 732 of the secondary strap 730 is attached to the lateral side of the ankle brace 700 near an upper edge or end. The distal end 732 of the secondary strap 730 may be positioned over the distal end of the dial strap 720 and may firmly hold the dial strap 720 to the brace body 702, or reinforce the attachment of the dial strap 720 to the brace body 702. In some instances, the wrapping of the dial strap 720 and secondary strap 730 may be reversed so that the secondary strap 730 is initially wrapped around the ankle brace 700 and the dial strap 720 is subsequently wrapped over the secondary strap 730.

The secondary strap 730 and/or the dial strap 720 may be formed of an elastic or semi-elastic material, or either or both of these straps may be formed of a non-elastic material. In some embodiments, the dial strap 720 and secondary strap 730 are opposing ends or sections of the same strap. The dial strap 720 and/or secondary strap 730 may be coupled with the ankle brace 700 so as to be at or below the wearer's foot.

Tensioning of the tension member 222 causes the lateral side and medial side to be pulled toward one another, which constricts the ankle brace 700 about the wearer's ankle. The dial strap 720 and secondary strap 730 may then be wrapped about the brace body 702 and fastened together according to the wearer's preference. The straps, 720 and 730, may be fastened via hook and loop fasteners, clamps, buckles, snaps, magnetic fastening, or using any other means of mechanism fastening.

While several embodiments and arrangements of various components are described herein, it should be understood that the various components and/or combination of components described in the various embodiments may be modified, rearranged, changed, adjusted, and the like. For example, the arrangement of components in any of the described embodiments may be adjusted or rearranged and/or the various described components may be employed in any of the embodiments in which they are not currently described or employed. As such, it should be realized that the various embodiments are not limited to the specific arrangement and/or component structures described herein.

In addition, it is to be understood that any workable combination of the features and elements disclosed herein is also considered to be disclosed. Additionally, any time a feature is not discussed with regard in an embodiment in this disclosure, a person of skill in the art is hereby put on notice that some embodiments of the invention may implicitly and specifically exclude such features, thereby providing support for negative claim limitations.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the device” includes reference to one or more devices and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

1. A reel based closure device comprising:
a housing;
a spool rotatably positioned within the housing, the spool comprising a channel formed therein about which a tension member is wound as the spool is rotated in a tightening direction and is unwound as the spool is rotated in a loosening direction;
a knob supported by the housing, the knob being rotatable with respect to the housing and being operably coupled to the spool such that rotation of the knob causes the spool to rotate in the tightening direction; and
a pawl component that is separate from the knob and operably couplable thereto, the pawl component including one or more pawls comprising a pawl arm having a proximal end that is coupled to the pawl component and a distal end that includes one or more pawl teeth; wherein:
the pawl component includes a lip or wall; and
the pawl arm includes a projection or tab that engages with the lip or wall of the pawl component to control radial movement of the pawl arm relative to the pawl component.

2. The reel based closure device of claim 1, wherein the pawl component is disc shaped.

3. The reel based closure device of claim 1, wherein the lip or wall is formed or defined on outer peripheral edge of pawl component.

4. The reel based closure device of claim 1, wherein the projection or tab is formed on a radially interior edge of pawl arm.

5. The reel based closure device of claim 4, wherein the projection or tab is positioned on the distal end of the pawl arm.

6. The reel based closure device of claim 1, wherein the one or more pawls engage with teeth when the knob is in an engaged position, and wherein the one or more pawls disengage from the teeth when the knob is in a disengaged position.

7. The reel based closure device of claim 6, wherein the knob is moveable axially with respect to the housing between the engaged position and the disengaged position.

8. The reel based closure device of claim 1, wherein the proximal end of the pawl arm is fixedly coupled to the pawl component.

9. A reel based closure device comprising:
a housing;
a spool rotatably positioned within the housing;
a knob operably coupled to the spool such that rotation of the knob causes the spool to rotate; and
a pawl component that includes one or more pawl arms having a proximal end that is coupled to the pawl component and a distal end that includes one or more pawl teeth; wherein:
the pawl component includes a lip or wall; and
the pawl arm includes a projection or tab that engages with the lip or wall of the pawl component to control radial movement of the pawl arm relative to the pawl component.

10. The reel based closure device of claim 9, wherein the pawl component is disc shaped.

11. The reel based closure device of claim 9, wherein the lip or wall is formed or defined on outer peripheral edge of pawl component.

12. The reel based closure device of claim 9, wherein the projection or tab is formed on a radially interior edge of pawl arm.

13. The reel based closure device of claim 12, wherein the projection or tab is positioned on the distal end of the pawl arm.

14. The reel based closure device of claim 9, wherein the one or more pawl teeth engage with teeth of the reel based closure device when the knob is in an engaged position, and wherein the one or more pawl teeth disengage from the teeth of the reel based closure device when the knob is in a disengaged position.

15. The reel based closure device of claim 14, wherein the knob is moveable axially with respect to the housing between the engaged position and the disengaged position.

16. The reel based closure device of claim 9, wherein the proximal end of the pawl arm is fixedly coupled to the pawl component.

17. A method of assembling a reel based closure device, the method comprising:
obtaining a housing;
positioning a spool within the housing so that the spool is rotatable within the housing;
coupling a pawl component with the housing, the pawl component including one or more pawl arms having a proximal end that is coupled to the pawl component and a distal end that includes one or more pawl teeth, wherein the pawl component includes a lip or wall and the pawl arm includes a projection or tab that engages with the lip or wall of the pawl component to control radial movement of the pawl arm relative to the pawl component; and
coupling a knob with the housing so that the knob is operably coupled to the spool.

18. The method of claim 17, further comprising positioning the projection or tab of the pawl arm radially inward of the lip or wall of the pawl component so that a radially outward movement of the pawl arm engages the projection or tab of the pawl arm with the lip or wall of the pawl component.

19. The method of claim 17, further comprising axially inserting the pawl component into a bottom end of the knob.

20. The method of claim 17, further comprising attaching the reel based closure device to an article of footwear.

21.-100. (canceled)

101. The reel based closure device of claim 1, wherein the reel based closure device is attached to a brace.

102. The reel based closure device of claim 9, wherein the reel based closure device is attached to a brace. ankle brace.

103. The method of claim 20, wherein the article of footwear is an
